(19) United States
(12) Patent Application Publication
(10) Pub. No.: US 2014/0107527 A1
(43) Pub. Date: Apr. 17, 2014

(54) UNIVERSAL MEASURING MODULE FOR MEDICAL APPLICATION AND METHOD FOR ASSEMBLING THE MEASURING MODULE

(71) Applicants: Gerrat Dijkman, Enschede (NL); Marc Herman Padberg, Enschede (NL)

(72) Inventors: Gerrat Dijkman, Enschede (NL); Marc Herman Padberg, Enschede (NL)

(73) Assignee: BEST MEDICAL B.V., Enschede (NL)

(21) Appl. No.: 14/004,393

(22) PCT Filed: Mar. 9, 2012
(86) PCT No.: PCT/NL2012/050146

§ 371 Date: Dec. 17, 2013

(30) Foreign Application Priority Data
Mar. 11, 2011 (NL) 2006377

(51) Int. Cl. A61B 5/20; H05K 3/30

(52) U.S. Cl. CPC A61B 5/205; H05K 3/30
USPC 600/561; 29/832

(57) ABSTRACT

The invention relates to a measuring module for medical application which is provided with a housing comprising a number of first connections for catheters, a number of pressure sensors, a number of conduits for fluid connection to one of the first connections, wherein each of the conduits comprises at least one opening for the purpose of fluid connection to one of the pressure sensors, and at least one second connection for a communication module for transfer of measurement data from the pressure sensors to the communication module. The measuring module has the feature that at least one gasket, which leaves the openings clear, is arranged between the conduits and the pressure sensors. The invention also relates to a method for assembling a measuring module according to the invention.

The invention relates to a universal measuring module for medical application and a method for assembling the measuring module.

The invention relates more particularly to a measuring module for medical application which is provided with a housing, comprising: a number of first connections for catheters, a number of pressure sensors, a number of conduits for fluid connection to one of the first connections, wherein each of the conduits comprises at least one opening for the purpose of fluid connection to one of the pressure sensors; and at least one second connection for a communication module for transfer of measurement data from the pressure sensors to the communication module.

Such a measuring module for medical application is per se known in the field. The known measuring module is particularly suitable for performing in vivo urodynamic pressure measurements. The measuring module is intended for once-only use. Since only the known measuring module comes into contact with fluids of a patient and the communication module does not, the know measuring module has to be discarded after use while the communication module is suitable for re-use.

The invention has for its object to provide such a measuring module, which is simpler and less expensive to produce than the known measuring module and from an economic viewpoint is therefore even more suitable as disposable article.

The measuring module according to the invention has for this purpose the feature that at least one gasket, which leaves the openings clear, is arranged between the conduits and the pressure sensors.

The method according to the invention is characterized by the following steps of:

a) arranging a gasket over the conduits such that the fluid openings remain clear;

b) arranging the pressure sensors on the gasket at the position of the fluid openings;

c) arranging the printed circuit board and mounting the pressure sensors on the printed circuit board.

By applying a gasket between the conduits and the pressure sensors in accordance with the inventive concept, the pressure sensors need no longer be adhered in the housing. The placing of a gasket can be performed much more quickly than forming an adhesive connection. Arranging the adhesive connection moreover regularly causes defects in the pressure sensors. The number of production errors can therefore be reduced by avoiding the adhesive connection.

In a first preferred embodiment the measuring module is further provided with a printed circuit board on which the pressure sensors are arranged, wherein an electrically conductive connecting element is arranged between each pressure sensor and the printed circuit board.

According to a first preferred embodiment of the method according to the invention, the method further comprises, after step b), the step of b1) arranging the electrically conductive connecting elements at the position of the pressure sensors; and step c1) comprises of arranging the printed circuit board over the electrically conductive connecting elements.

Application of electrically conductive connecting elements adapted to realize an electrical connection between the pressure sensors and the printed circuit board makes it possible to connect the pressure sensors to the printed circuit board without arranging the usual soldered connections. Arranging soldered connections is a time-consuming activity which must be performed very accurately. By means of the electrically conductive connecting elements the production of the measuring module according to the invention can take place not only more quickly, but also more accurately and so with fewer production errors.

Both the gasket and the electrically conductive connecting elements make it possible to releasably connect the pressure sensors and the components connected thereto. In addition to its releasability, this connection also has a certain tolerance. This is because the gasket material and the material of the electrically conductive connecting elements are more flexible than respectively the rigid adhesive connection and the soldered connection, so that these connections can be made more easily, also by less skilled personnel.

The measuring module according to the invention is universally suitable for fluid catheters, i.e. for all types of liquid catheters as well as for all types of gas catheters, including air catheters. The measuring module according to the invention preferably comprises a closing tap and/or a non-return valve and/or a vent tap.

The invention will now be described in more detail with reference to the figures.

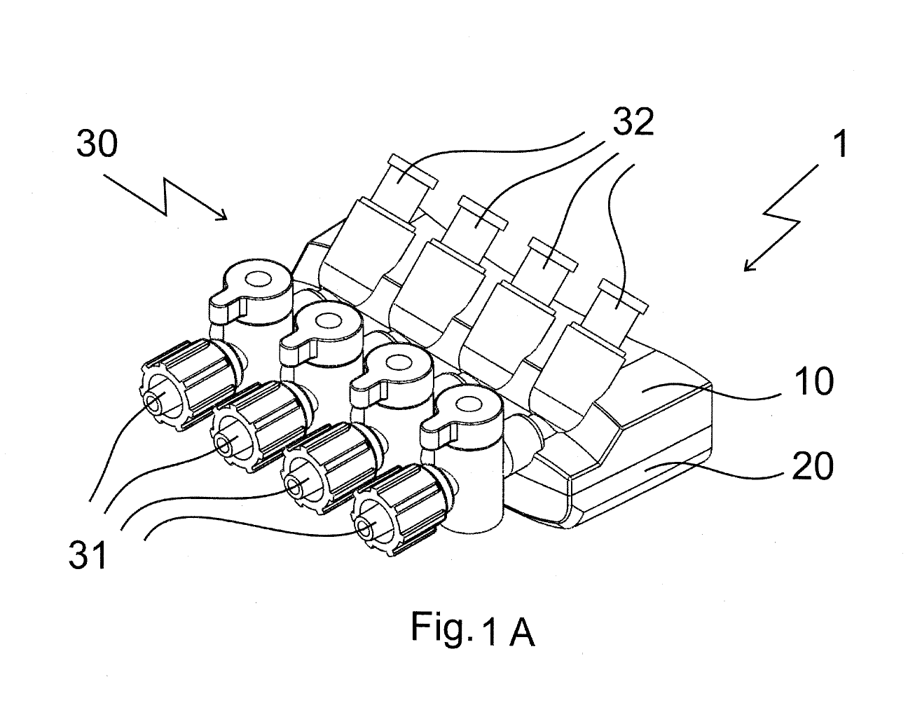

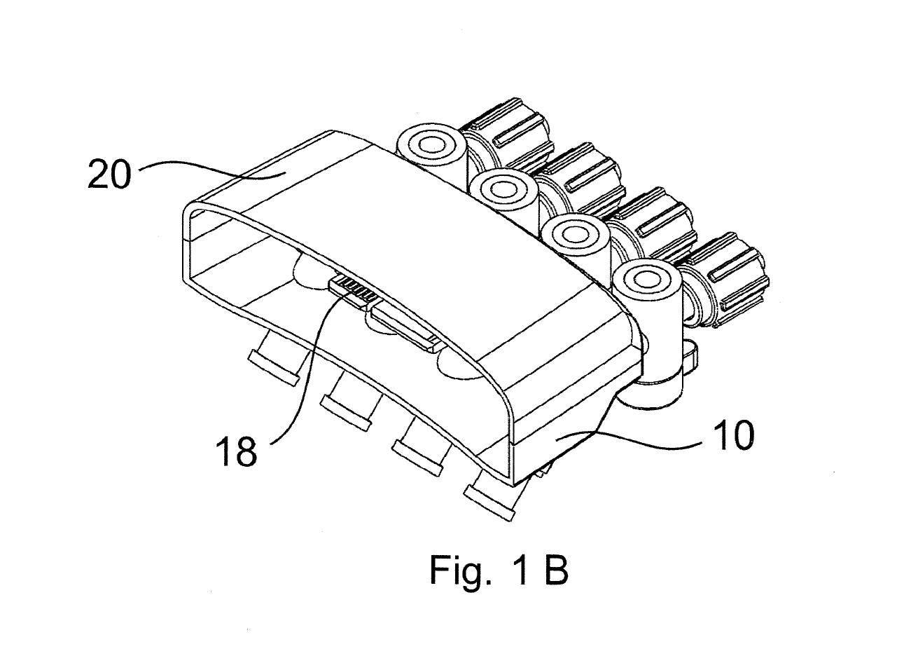

FIGS. 1A and 1B show a schematic view of a preferred embodiment of the universal measuring module according to the invention from different viewpoints;

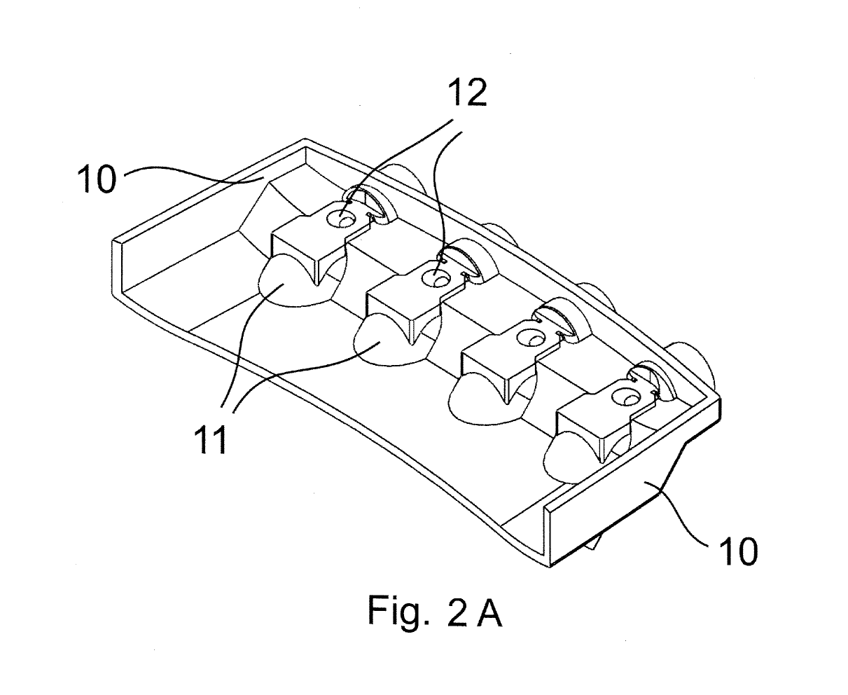

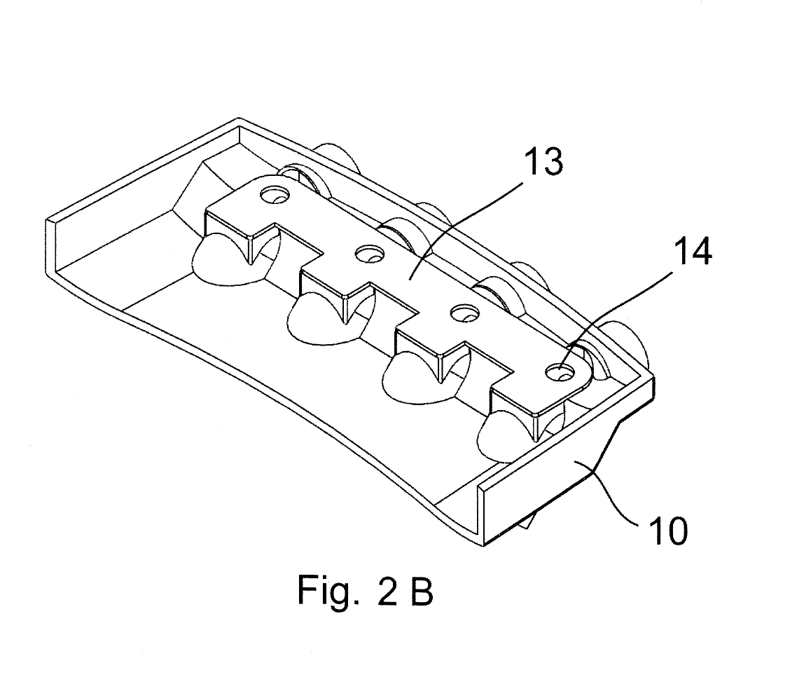

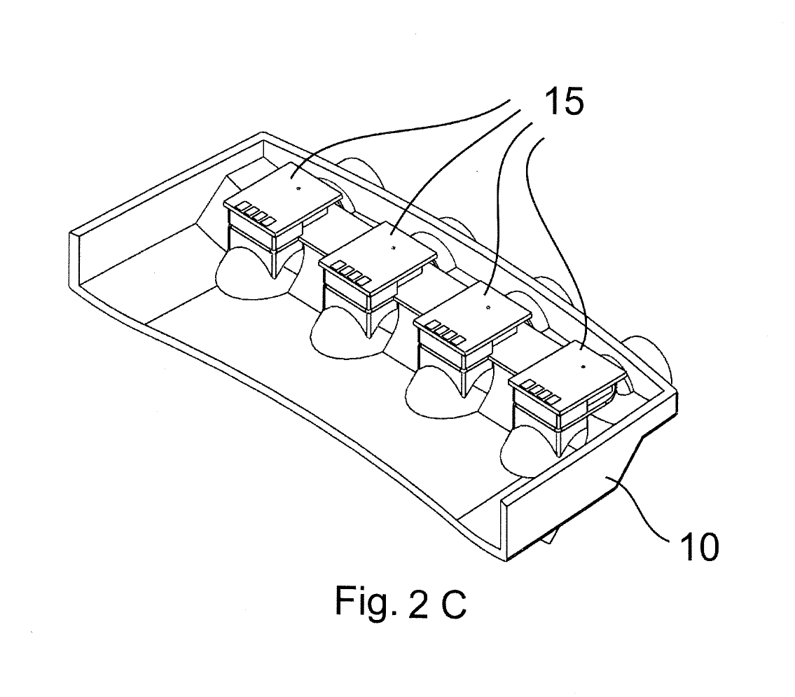

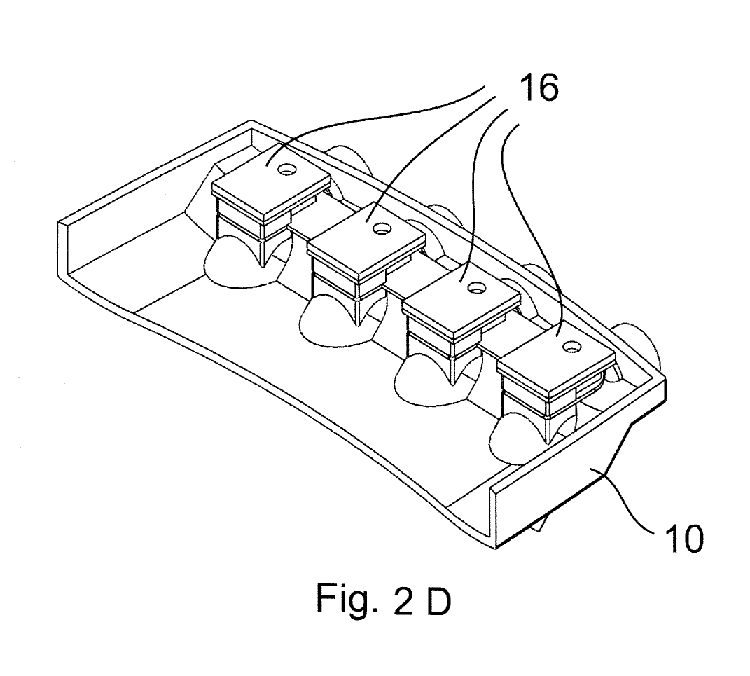

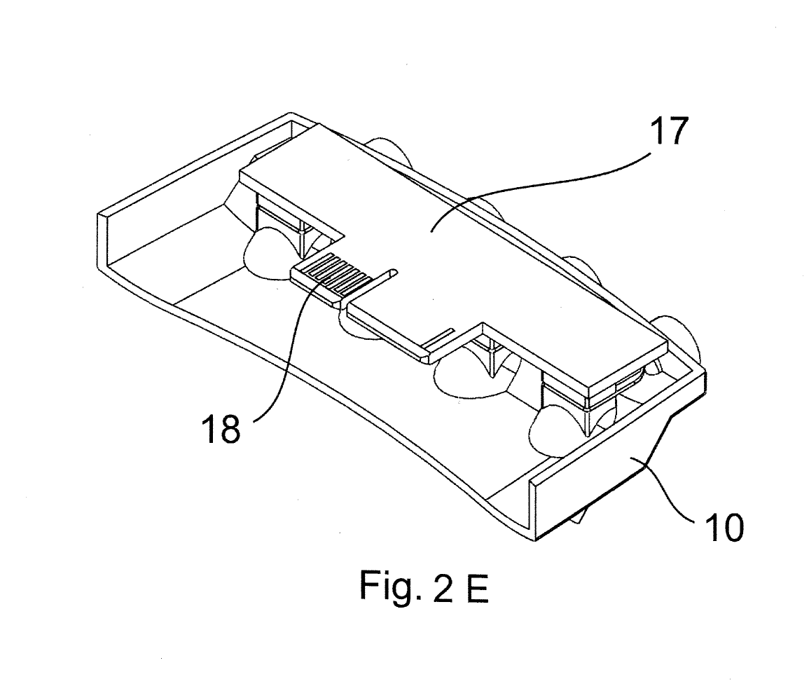

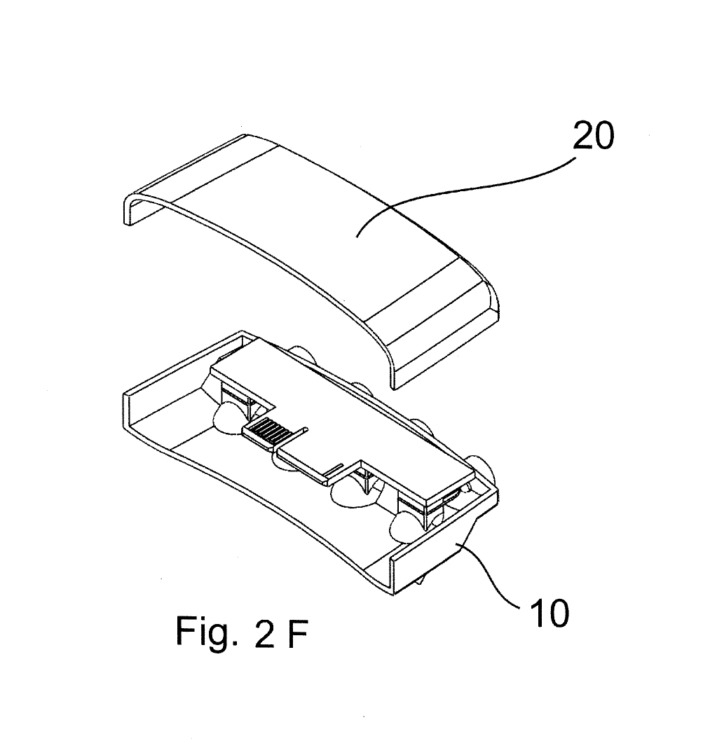

FIGS. 2A to 2F show the universal measuring module of FIGS. 1A and 1B at different stages of the assembly performed in accordance with the method according to the invention; and

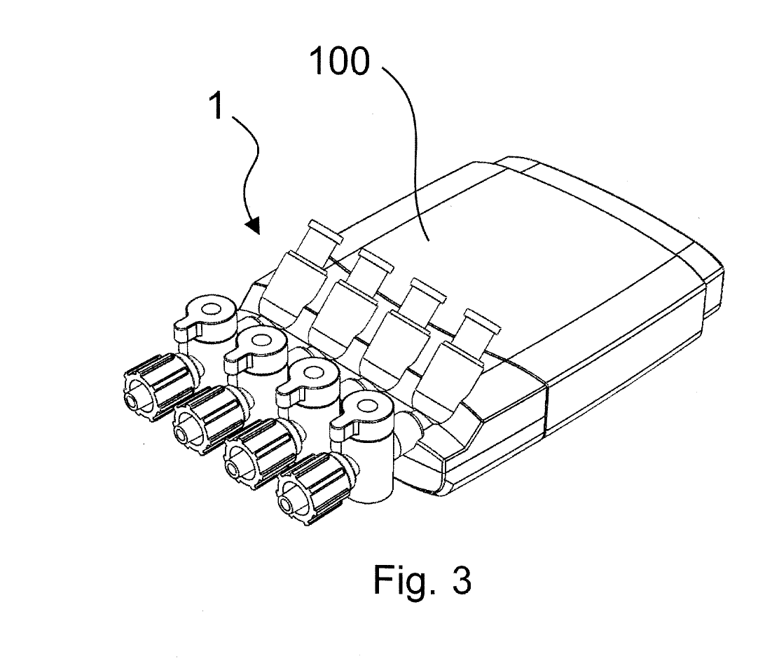

FIG. 3 shows a schematic view of a system of a communication module and the universal measuring module according to the invention.

The same components are provided in all figures with the same reference numerals.

FIG. 1A and FIG. 1B show a universal measuring module 1 according to the invention. The universal measuring module 1 is shown in a preferred embodiment in which it consists of two housing parts, i.e. a first housing part 10 and a second housing part 20. In use the first housing part 10 forms the upper side of the universal measuring module 1 and the second housing part 20 forms the underside of the universal measuring module 1. First connections 30 are arranged on module 1 for connection of liquid catheters and/or air catheters. Received in the housing, which can also be manufactured integrally, are second connecting means 18 for connection to a further module. This further module is preferably a communication module for receiving measurement data from module 1 and transmitting these measurement data to peripheral equipment for further processing of the measurement data.

FIGS. 2A to 2F illustrate measuring module 1 in the preferred embodiment at different stages of assembly. FIG. 3 shows a system of a universal measuring module 1 according to the invention with a communication module 100.

The method for assembling measuring module 1 according to the invention will now be elucidated with reference to FIGS. 2A to 2F.

FIG. 2A shows the first housing part 10. Arranged herein are a number of conduits 11 are which are each in fluid connection with one of the catheter connections 30. Each of the conduits 11 comprises a fluid opening 12. Housing part 10 is preferably embodied as injection-moulded part.

Relative to FIG. 2A, a gasket 13 is arranged over conduits 11 in FIG. 2B. Gasket 13 serves substantially as air seal and liquid seal. Gasket 13 is embodied such that it leaves fluid openings 12 clear. In the shown preferred embodiment gasket 13 is provided for this purpose with gasket apertures 14. Gasket 13 is preferably manufactured from a soft material. Diverse suitable materials are known for gasket 13. Several examples are polyurethane and silicone.

In FIG. 2C pressure sensors 15 are arranged at the position of gasket apertures 14. A pressure sensor 15 is now situated on each of the conduits 11. Suitable pressure sensors are known in the relevant field. An example of a suitable pressure sensor is an MSI sensor or a Motorola MPX 2300DT1.

In FIG. 2D intermediate electrically conductive connecting elements 16 arranged on pressure sensors 15. These connecting elements 16 serve to bring about an electrical connection between pressure sensors 15 and printed circuit board 17, which has to be arranged over connecting elements 16 and gasket 13 and pressure sensors 15 (see in FIG. 2E). The electrically conductive connecting elements 16 are preferably manufactured from an electrically conductive plastic. Suitable electrically conductive connecting elements 16 are known in the relevant field for application in high-grade technical production processes of LCD displays and processors. A number of examples of a suitable type of connecting element are the Inter-Connectors marketed by Shin-Etsu Polymer Co. Ltd, for instance of the MS-G or SEC type.

FIG. 2E shows schematically the position of printed circuit board 17. In the shown preferred embodiment printed circuit board 17 is provided with a second connection 18. This connection 18 is intended for the purpose of connecting measuring module 1 to the communication module shown in FIG. 3. Printed circuit board 17 more preferably comprises a programmable memory (not shown) adapted to guarantee once-only use of measuring module 1.

Printed circuit board 17 is preferably mounted on housing part 10, for instance by means of a screw connection, a snap connection or an adhesive connection.

In FIG. 2F the second housing part 20, which serves to close measuring module 1, is arranged. The second housing part 20 co-acts with the first housing part 10 in order to form a closed housing. Housing part 10 is preferably embodied as injection-moulded part.

The connection between the first housing part 10 and the second housing part 20 can be realized in different ways. Some examples are: by means of a snap connection, an adhesive connection or an (ultrasonic) welded connection.

FIG. 3 shows a schematic view of a system of a communication module 100 to which the universal measuring module 1 according to the invention is connected.

The first connections 30 for the catheters are finally arranged. The connections are preferably such that the catheters can be releasably mounted. In the shown preferred embodiment connections 30 comprise closing taps 31 and non-return valves 32.

The measuring module according to invention is described in the context of urological applications. It will be apparent to a skilled person in the field that the measuring module is also suitable for performing in vivo pressure measurements in other medical fields, such as blood pressure measurements or gastro-enterological measurements.

The invention is of course not limited to the described and shown preferred embodiments but extends to any embodiment falling within the scope of protection as defined in the claims and as seen in the light of the foregoing description and accompanying drawings.

1. A measuring module for medical application which is provided with a housing (10; 20), comprising
a number of first connections (30) for catheters,
a number of pressure sensors (15),
a number of conduits (11) for fluid connection to one of the first connections (30), wherein each of the conduits comprises at least one opening (12) for the purpose of fluid connection to one of the pressure sensors (15); and
at least one second connection (18) for a communication module for transfer of measurement data from the pressure sensors to the communication module, characterized in that at least one gasket (13), which leaves the openings (12) clear, is arranged between the conduits (11) and the pressure sensors (15).

2. A measuring module as claimed in claim 1, wherein the measuring module (1) is further provided with a printed circuit board (17) on which the pressure sensors (15) are arranged, wherein an electrically conductive connecting element (16) is arranged between each pressure sensor (15) and the printed circuit board (17).

3. A measuring module as claimed in claim 2, wherein the at least one second connection (18) is arranged on the printed circuit board (17).

4. A measuring module as claimed in claim 1, wherein the first connections (30) are adapted for releasable connection of the catheters.

5. A measuring module as claimed in claim 1, wherein each first connection comprises a closing tap (31).

6. A measuring module as claimed in claim 1, wherein each first connection comprises a non-return valve (32).

7. A measuring module as claimed in claim 1, wherein each first connection comprises a vent tap.

8. A method for assembling a measuring module as claimed in claim 1, characterized by the following steps of:
a) arranging a gasket (13) over the conduits (11) such that the fluid openings (12) remain clear;
b) arranging the pressure sensors (15) on the gasket (13) at the position of the fluid openings (12); and
c) arranging the printed circuit board (17) and mounting the pressure sensors (15) on the printed circuit board.

9. The method as claimed in claim 8 for assembling a measuring module according to claim 2, wherein the method further comprises, after step b), the step of:
b1) arranging the electrically conductive connecting elements (16) at the position of the pressure sensors (15); and wherein step c) comprises of
c1) arranging the printed circuit board (17) over the electrically conductive connecting elements (16)
